(19) United States
(12) Patent Application Publication
(10) Pub. No.: US 2007/0253914 A1
(43) Pub. Date: Nov. 1, 2007

(54) Method of exfoliation

(76) Inventors: Robert Bao Kim Ha, Liberty Twp, OH (US); Melissa Shrock Robison, Milford, OH (US); Timothy John Walter Cilley, Mason, OH (US)

Correspondence Address: THE PROCTER & GAMBLE COMPANY;INTELLECTUAL PROPERTY DIVISION - WEST BLDG., WINTON HILL BUSINESS CENTER - BOX 412, 6250 CENTER HILL AVENUE, CINCINNATI, OH, 45224, US

(21) Appl. No.: 11/414,084

(22) Filed: Apr. 28, 2006

(51) Int. Cl. A61K 8/72; A61K 8/64; A61K 8/97

(52) U.S. Cl. 424/47; 424/70.17; 424/70.11; 424/74; 424/70.14

(57) ABSTRACT

Method of exfoliating mammalian skin comprising the step of using a foam applicator to apply a personal care composition to delicate mammalian skin, wherein the foam applicator comprises a material selected from the group consisting of polyester, polyurethane, polyether, polyethylene, polystyrene, cross-linked polyethylene, and mixtures thereof, and has a porosity of from about 10 to about 110 pores per square inch; and wherein the personal care composition comprises a dermatologically-acceptable carrier in the form of an emulsion.

### FIELD OF THE INVENTION

The present invention relates to methods of exfoliation of delicate mammalian skin.

### BACKGROUND OF THE INVENTION

Exfoliation is thought to provide a number of benefits to skin, including softer, healthier skin and a more youthful appearance. A number of products are available to consumers to aid in exfoliation, for example scrubs, masks and implements. Such products typically are not suitable for use on the delicate skin such as the skin surrounding the eye. The product instructions often indicate to avoid usage in the eye area, and consumers furthermore may be reluctant to exfoliate near the eye for fear of discomfort or of damaging the skin. Nevertheless, exfoliation of skin surrounding the eye may be beneficial for a number of reasons. Removal of excess dead skin cells and debris may increase circulation. The area surrounding the eye is rich in blood vessels; therefore, increasing circulation in this area may be particularly beneficial to increase delivery of actives and to decrease puffiness resulting from fluid retention. A need exists, therefore, to provide a means for gentle exfoliation of the skin surrounding the eyes.

In addition to exfoliation, it also is desirable to provide a means for improving the condition of skin around the eyes, for example, by delivery of benefit agents that provide acute and/or chronic benefits. Furthermore, when compositions are applied to the eye area, it is desirable to provide a means for convenient, controlled delivery to prevent, for example, the product from getting into the eyes. A continuing need exists, therefore, to provide a means for convenient delivery of skin benefit agents to the eye area.

### SUMMARY OF THE INVENTION

The present invention meets the aforementioned needs and describes a method of exfoliation suitable for delicate skin, comprising the step of applying a personal care composition in the form of an emulsion by means of a suitable foam applicator. Previously, it was thought that applying an oil-containing emulsion to a foam applicator would render the foam unsuitable for exfoliation. Applicants have found, however, that an exfoliation benefit is realized beyond that which would be expected from use of the foam applicator or the composition alone. Applicants further believe that by delivering the composition simultaneously with exfoliation, penetration of skin care actives is increased. This may improve delivery of actives especially suited to delicate skin, including the skin surrounding the eye.

The foam applicator may form part of a container for the composition. In one embodiment, the container is a disposable pouch, suitable for a single dose application. Such an applicator provides the additional advantages of a means to easily store and transport skin care compositions, for example in a purse or while traveling. In addition, application is relatively hygienic, as the need to touch the eye area with fingers or with contaminated implements is eliminated.

The following describe some non-limiting embodiments of the present invention.

According to the first embodiment of the present invention, a method of exfoliating mammalian skin is provided, comprising the step of applying a personal care composition to delicate mammalian skin by means of a foam applicator. The applicator comprises materials selected from the group consisting of polyurethane, polyester, polyether, polyethylene, polystyrene, cross-linked polyethylene, and mixtures thereof, and has a porosity of from about 3 to about 110 pores per linear inch. The personal care composition is an emulsion, and may comprise a particulate material. The applicator may be stably affixed to a container, which may comprise an amount of composition suitable for a single application. The method further may include a means for indicating that the composition is dispensed onto the applicator.

According to yet another embodiment of the present invention, a method of exfoliating mammalian skin and providing at least one additional benefit to the skin is provided, comprising the step of applying a personal care composition comprising at least one skin benefit agent to delicate mammalian skin, by means of the applicator described in the first embodiment. The benefit agent may be a chronic and/or an acute benefit agent.

According to yet another embodiment of the present invention, an article of commerce is provided, comprising a personal care composition and a foam applicator as described in the first embodiment, and a communication directing a consumer to apply the composition to delicate skin to provide an exfoliation benefit.

According to yet another embodiment of the present invention, a kit is provided, comprising a personal care composition and a foam applicator, as described in the first embodiment, and a plurality of components including, but not limited to, one or more additional compositions, one or more orally ingestible dietary supplements, an additional implement, an additional delivery enhancement device, a temperature change element, a substrate, instructions for complying with suitable application regimens, and combinations thereof.

### DETAILED DESCRIPTION OF THE INVENTION

The present invention describes a method of exfoliation of mammalian skin comprising the step of applying a suitable composition to the skin with a foam applicator. The applicator may be used on essentially any type of mammalian skin, and is particularly suitable for use on delicate skin, including skin surrounding the eyes, and on other sensitive skin. The present invention further describes a method for simultaneously exfoliating and providing an additional benefit to mammalian skin comprising the step of applying a composition comprising at least one skin care active to delicate skin with a foam applicator.

In all embodiments of the present invention, all percentages are by weight of the total composition, unless specifically stated otherwise. All ratios are weight ratios, unless specifically stated otherwise. All ranges are inclusive and combinable. The number of significant digits conveys neither limitations on the indicated amounts nor on the accuracy of the measurements. All numerical amounts are understood to be modified by the word “about” unless otherwise specifically indicated. All measurements are understood to be made at 25° C. and at ambient conditions, where “ambient conditions” means conditions under about one atmosphere of pressure and at about 50% relative humidity.

“Personal care composition,” as used herein, means a composition that may be applied to mammalian keratinous tissue without undue undesirable side effects.

“Keratinous tissue,” as used herein, means keratin-containing layers disposed as the outermost protective covering of mammals and includes, but is not limited to, skin, hair and scalp.

“Skin surrounding the human eye,” or grammatical equivalents thereof, is understood to mean skin of the regio orbitalis, or orbital region, and includes the skin covering all or part of the musculus orbicularis, as defined in Sobotta Atlas of Human Anatomy, Vol. 1, 10^{th} English Edition, 1982. This area includes skin covering the eyelid, the eyelid, undereye area, and the inner and outer corners of the eyes.

“Delicate skin,” as used herein, means skin having an average thickness approximately equal to the average thickness of skin covering a human eyelid, e.g. from about 20 micrometers to about 35 micrometers; skin which is essentially disconnected from an underlying bony structure; which has limited elasticity, understood to mean the ability of the skin to return to its original position without physical assistance after being shifted relative to the underlying tissue; skin surrounding the human eye; and/or sensitive skin. Non-limiting examples of sensitive skin include skin on which scrubbing, repeated rubbing or other similar mechanical forces result in undue discomfort or irritation, for example skin on the inner arms or thighs, diseased and/or damaged skin, and skin exposed to allergens.

“Exfoliation,” as used herein, means removal of keratinocytes from the surface of mammalian skin, by mechanical and/or chemical means, without causing undue discomfort and/or damage to the skin, including but not limited to irritation, redness, abrasion, etc. Exfoliation may be evidenced by the presence of stained skin cells, made visible to the unaided eye by methods that would be known by one of skill in the art, on the foam applicator after use as described herein. Alternatively, exfoliation may be shown by the following method: Apply approximately 50 microliters of a composition comprising 5% DHA to two separate areas of skin measuring approximately 3 cm×3 cm on the volar forearm once in the morning and once in the afternoon on Day 1. On Day 2, obtain a baseline color measurement of both areas using a MINOLTA® CR-300 Chroma Meter, which generates values for L, a, and b, described by Robertson, A. R. in “The CIE 1976 Color Difference Formulas,” Color Research Applications, vol. 2, pp. 7-11 (1977). Apply about 0.5 ml of the composition with the applicator, both described herein, once per day on Days 2, 3, and 4 at a dose of about 0.5 ml, to one area of skin, rubbing gently in a circular motion for 30 seconds. After each application, obtain a color measurement of L, a, and b values for the area of skin to which the composition has been applied, and for the area of skin to which no composition has been applied. Obtain a final color measurement for both areas on Day 5, approximately 24 hours after the last application of the composition. Calculate the average of the b values. The occurrence of exfoliation may be inferred when the average of the b-values for the two areas of skin are statistically significantly different at the 95% confidence level.

“Benefit,” as used herein in reference to mammalian skin, means a benefit to the skin in addition to exfoliation. The benefit may result directly or indirectly from exfoliation, and/or be enhanced by exfoliation, and may be acute or chronic. Benefits include regulating and/or improving the condition of mammalian skin. Herein, “regulating skin condition” means maintaining desirable skin qualities, such as appearance and/or feel, or delaying the onset of undesirable skin qualities. Herein, “improving skin condition” means effecting a visually and/or tactilely perceptible positive change in skin appearance and feel. Some non-limiting examples of benefits include delaying the onset and/or reducing the appearance of lines, wrinkles and/or unevenness; thickening of the skin; increasing the convolution of the dermal-epidermal border (also known as the rete ridges); improving skin elasticity; delaying the loss of skin elasticity; reducing the appearance of discoloration to the skin, for example, under-eye circles, blotchiness (e.g., uneven red coloration due to, for example, rosacea), sallowness, etc.; reducing puffiness and/or swelling; alleviating chronic and/or acute dry skin; reducing the appearance of hyperpigmented skin regions such as age spots and freckles; etc.

“Skin benefit agents,” or alternatively “skin care actives,” as used herein, means compounds that, when applied to the skin, provide an acute and/or chronic benefit to keratinous tissue, including the skin.

“Visibly distinct,” as used herein in reference to the composition relative to the applicator, means that the composition is distinguishable from the applicator by the human eye, on the basis of color, without the aid of magnification.

“Stably affixed,” as used herein in reference to the foam applicator and a container, means that the applicator and the container form a sealed unit, such that the composition remains within the container under reasonable transport and storage conditions. When pressure is applied, for example by squeezing with the fingers, all or part of the composition may be transferred from the container to the applicator, so as to allow reasonably uniform distribution to the skin.

“Controlled flow,” as used herein, means that upon application of light pressure to a container (for example, squeezing with the fingers), the composition contained therein flows selectively onto the applicator, and is retained until the applicator is applied to the skin, without the composition dripping, flowing, running, etc. off of the applicator.

“Reasonably uniform distribution,” means that when the applicator is placed in contact with the skin under relatively constant pressure, the rate of transfer of the composition from the applicator to the skin also is relatively constant, i.e. is not characterized by a sudden flow or gush of the composition from the applicator.

“Simultaneously,” as used herein in reference to exfoliation and providing a benefit, means that the exfoliation and application of the benefit agent occur in a single step, for example, by applying the composition with the applicator. Simultaneously should not be understood to mean that the exfoliation and the manifestation of the benefit necessarily occur at the same time.

I. Applicator

The applicator of the present invention is a foam applicator, suitable for exfoliating mammalian skin, and in particular, the skin surrounding the eyes and other delicate mammalian skin. The foam may comprise polyester, polyurethane, polyether, polyethylene, polystyrene, cross-linked polyethylene and mixtures thereof. In one embodiment, the foam comprises polyurethane. The thickness and porosity of the foam may vary, and are suited to the viscosity of the composition, in that the composition should be sufficiently retained on the applicator so as to allow reasonably uniform distribution onto the skin. For example, the composition should remain on the applicator until the applicator is placed in contact with the skin without dripping or running, and should transfer easily to the skin upon gentle wiping. The foam is an open-cell foam, may be reticulated foam, and may be coated, for example, with polyvinyl chloride or with other suitable coating materials. Alternatively, the foam may be compressed, reticulated felt foam. Non-limiting examples of suitable foams are commercially available from Foamex Technical Products (Eddystone, Pa.), and include SIF®, SIF®-PVC, and SIF Felt® foams. The foam applicator of the present invention is understood not to include foam which causes a scratching or abrasive sensation when moved across the skin using light pressure, or which leaves visible markings on delicate skin after use as described herein.

When the foam is other than compressed, reticulated felt foam, the foam has a porosity of from about 3 to about 110, alternatively from about 30 to about 110, and alternatively of about 80 to about 110, pores per linear inch (PPI). Porosity may be correlated to air flow, as measured by ASTM method D3574. When the foam is a compressed, reticulated felt foam, the foam may have an uncompressed density of from about 5 to about 30 pounds per cubic foot (lbs/ft³), also as measured by ASTM method D3574. In one embodiment, the foam may have an uncompressed thickness of about 1 mm to about 8 mm, alternatively from about 3 to about 5 mm, and alternatively of about 4 mm.

The foam applicator may have a variety of shapes and sizes, suitable for the intended application. In one embodiment, the foam applicator is round, and has a diameter of from about 1 cm to about 5 cm, and alternatively of from about 2 to about 3 cm. Alternatively, the foam applicator may be oval shaped. Alternatively, the foam applicator may have at least one elongated region, for example, a pointed or finger-shaped region, suitable for accessing small or difficult to reach areas (for example, the corners of the eyes).

The foam applicator may have a variety of colors. In one embodiment, the foam applicator is white. Alternatively, the applicator is visibly distinct from the composition, and thus provides a means for indicating that the composition is dispensed onto the applicator.

The foam applicator may be stably affixed to a container, suitable for storing and for dispensing the composition. When the applicator is affixed to a container, the porosity of the foam, the thickness of the foam, and the viscosity of the composition are suitable to allow the composition to be dispensed without rupturing the container, the foam, and/or a seal between the foam and the container, and furthermore are suitable to allow reasonably uniform distribution onto the skin. The container may be semi-rigid, flexible, or collapsible. In one embodiment, the container comprises an amount of a composition suitable for a single application to an area of mammalian skin (a “unit dose”), for example, the area of skin surrounding human eyes. In one embodiment, the container comprises from about 0.1 ml to about 10 ml, alternatively from about 0.1 ml to about 5 ml, alternatively from about 0.1 ml to about 1 ml, and alternatively about 0.5 ml of a suitable composition. One non-limiting example of a suitable container is the Delpouch™ (Cardinal Health), exemplified in U.S. Pat. No. 6,007,264, optionally modified to fulfill the above-described conditions relating to the applicator and to application of the composition to the skin.

II. Composition

The personal care composition of the present invention comprises a dermatologically-acceptable carrier in the form of an emulsion and may comprise at least one acute and/or chronic skin benefit agent. One non-limiting example of an acute skin benefit agent is a particulate material. The composition may have a viscosity of from about 20,000 centipoise (cps) to about 100,000 cps, alternatively from about 45,000 cps to about 90,000 cps, and alternatively is about 55,000 cps to about 60,000 cps. The viscosity of the composition may vary, and may depend upon the porosity of the applicator. Viscosity can be determined using a Brookfield RVDV-II digital viscometer, a T-C spindle (Spindle 93, 27.1 mm crossbar length), at 5 rpm, or the equivalent thereof. Prior to viscosity measurement, the composition is allowed to stabilize following its preparation or any agitation which results from handling. Generally, stabilization should last at least 24 hours under conditions of 25° C. ±1° C. and ambient pressure. In further preparation for viscosity measurements, the compositions are placed in containers which will produce at most minimal frictional effects on the viscosity determination (e.g., a 2 oz. glass jar with an orifice of at least 28 mm). The viscosity is measured with the composition at a temperature of 25° C. ±1° C. and after 30 seconds of spindle rotation. Five (5) viscosity measurements are gathered and the mean of the measurements is calculated in order to determine the viscosity of the composition.

The composition may be colorless, white, colored, or combinations thereof. In one embodiment, the composition is colored and visibly distinct from the foam applicator. When applied and distributed evenly onto the skin, the composition may be visibly indistinguishable from the underlying skin.

The composition may be substantially free of compounds that may cause undue pain and/or irritation of the eyes upon direct contact with the eyes or application in the vicinity of the eyes. Such compounds include, but are not limited to, harsh surfactants, for example lauryl sulfates, lauryl ether sulfates; acids, including acidic exfoliants such as lactic acid, hydroxy acids, etc., particulate materials having an average diameter greater than about 50 microns; volatile eye irritants, etc. By “substantially free” is meant that whereas trace amounts may incidentally be present, the compound is not expressly added to the composition during manufacture. In one embodiment, “substantially free” is understood to mean that 0.001% and less of the compound is present.

The composition may be understood to not provide a significant cleansing benefit, meaning that although some impurities, for example, dirt and/or make-up, may incidentally be removed from the skin during use, the composition is not intended primarily for use as a cleanser, for example, a tonic or a rinse-off composition.

A. Carrier

The skin care composition of the present invention may comprise from about 50% to about 99.9% of a dermatologically acceptable carrier. The carrier of the present invention is in the form of an emulsion. Herein, “emulsions” generally contain an aqueous phase and an oil phase. The oils may be derived from animals, plants, or petroleum, may be natural or synthetic, and may include silicone oils. Emulsion carriers include, but are not limited to, oil-in-water, water-in-oil, water-in-oil-in-water, and oil-in-water-in-silicone emulsions. In one embodiment, the dermatologically acceptable carrier comprises an oil-in-water emulsion, and alternatively, a silicone-in-water emulsion. The emulsion further may comprise a humectant, for example, glycerin and a non-ionic, cationic and/or anionic emulsifier. Suitable emulsifiers are disclosed in, for example, U.S. Pat. No. 3,755,560 issued to Dickert et al., U.S. Pat. No. 4,421,769, issued to Dixon et al., and McCutcheon's Detergents and Emulsifiers, North American Edition, pages 317-324 (1986).

B. Particulate Material

The composition of the present invention may comprise particulate material. In one embodiment, the composition may comprise from about 0.1% to about 20%, alternatively from about 0.2% to about 10%, and alternatively from about 0.5% to about 5%, of one or more particulate materials. Whereas the particulate material may provide some exfoliation benefit, a more typical use is to provide a pleasant skin feel and/or some additional acute benefit, for example, UV-protection. The particulates can, for example, be platelet shaped, spherical, elongated or needle-shaped, or irregularly shaped, surface coated or uncoated, porous or non-porous, charged or uncharged, and can be added to the current compositions as a powder or as a pre-dispersion; however, the particulate material should be of a size and shape such that when applied as described herein, would not irritate and/or damage delicate skin. Suitable particulate materials may have a size of from about 0.1 micron to about 50 microns.

Some non-limiting examples of particulate materials useful in the present invention include colored and uncolored pigments, interference pigments, inorganic powders and organic powders, composite powders, optical brightener particles, and mixtures thereof. These particulate materials can be derived from natural and/or synthetic sources.

Suitable organic powders particulate materials include, but are not limited, to spherical polymeric particles chosen from the methylsilsesquioxane resin microspheres, for example, Tospearl™ 145A, (Toshiba Silicone); microspheres of polymethylmethacrylates, for example, Micropearl™ M 100 (Seppic); the spherical particles of crosslinked polydimethylsiloxanes, for example, Trefil™ E 506C or Trefil™ E 505C (Dow Corning Toray Silicone); sphericle particles of polyamide, for example, nylon-12, and Orgasol™ 2002D Nat C05 (Atochem); polystyrene microspheres, for example Dyno Particles, sold under the name Dynospheres™, and ethylene acrylate copolymer, sold under the name FloBead™ EA209 (Kobo); aluminum starch octenylsuccinate, for example Dry Flo™ (National Starch); microspheres of polyethylene, for example Microthene™ FN510-00 (Equistar), silicone resin, polymethylsilsesquioxane silicone polymer, platelet shaped powder made from L-lauroyl lysine, and mixtures thereof.

Also useful herein are interference pigments. Herein, “interference pigments” means thin, platelike layered particles having two or more layers of controlled thickness. The layers have different refractive indices that yield a characteristic reflected color from the interference of typically two, but occasionally more, light reflections, from different layers of the platelike particle. The most common examples of interference pigments are micas layered with about 50-300 nm films of TiO₂, Fe₂O₃, silica, tin oxide, and/or Cr₂O₃. Such pigments often are pearlescent. Pearlescent pigments reflect, refract and transmit light because of the transparency of pigment particles and the large difference in the refractive index of mica platelets and, for example, the titanium dioxide coating. Intereference pigments are available commercially from a wide variety of suppliers, for example, Rona (Timiron™ and Dichrona™), Presperse (Flonac™), Englehard (Duochrome™), Kobo (SK-45-R and SK-45-G), BASF (Sicopearls™) and Eckart (Prestige™). In one embodiment, the average diameter of the longest side of the individual particles of interference pigments is less than about 75 microns, and alternatively less than about 50 microns.

Other pigments useful in the present invention can provide color primarily through selective absorption of specific wavelengths of visible light, and include inorganic pigments, organic pigments and combinations thereof. Examples of such useful inorganic pigments include iron oxides, ferric ammonium ferrocyanide, manganese violet, ultramarine blue, and chromium oxide. Organic pigments can include natural colorants and synthetic monomeric and polymeric colorants. An example is phthalocyanine blue and green pigment. Also useful are lakes, primary FD&C or D&C lakes and blends thereof. Also useful are encapsulated soluble or insoluble dyes and other colorants. Inorganic white or uncolored pigments useful in the present invention, for example TiO₂, ZnO, or ZrO₂, are commercially available from a number of sources, for example, TRONOX TiO₂ series, SAT-T CR837, a rutile TiO2 (U.S. Cosmetics). Also suitable are charged dispersions of titanium dioxide, disclosed in U.S. Pat. No. 5,997,887, issued to Ha et al.

C. Other Skin Benefit Agents

The composition of the present invention may comprise at least one additional skin benefit agent, or skin care active, useful for improving the appearance and condition of mammalian skin. Whereas the skin benefit agents of the present invention may provide an acute benefit, for the purposes of the present invention, the following are considered long-term, or chronic, skin benefit agents. The additional skin benefit agent may provide some exfoliation benefit, and alternatively, the benefit of the skin care active may be enhanced by exfoliation, for example, when the active exhibits increased penetration due to exfoliation. Classes of suitable skin care actives include, but are not limited to vitamins, peptides and peptide derivatives, sugar amines, sunscreens and/or ultraviolet light absorbers, oil control agents, flavonoid compounds, antioxidants, preservatives, phytosterols, protease inhibitors, tyrosinase inhibitors, anti-inflammatory agents, and mixtures thereof. It should be noted, however, that many skin care actives may provide more than one benefit, or operate via more than one mode of action. Therefore, classifications herein are made for the sake of convenience and are not intended to limit the active to that particular application or applications listed.

1. Vitamins

The composition of the present invention may comprise one or more vitamins, for example, to provide antioxidant and/or other nutritional benefits to the skin. Herein, “vitamins” means vitamins, pro-vitamins, and their salts, isomers and derivatives. The vitamins may include water soluble vitamins, for example, vitamin B compounds (including B3 compounds such as niacinamide; nicotinic acid, C1-C18 nicotinic acid esters, and nicotinyl alcohol; B6 compounds, such as pyroxidine; and B5 compounds, such as panthenol, or “pro-B5”); and vitamin C compounds, including ascorbyl esters of fatty acids, and ascorbic acid derivatives, for example, ascorbyl glucoside, magnesium ascorbyl phosphate, sodium ascorbyl phosphate, and ascorbyl sorbate; and mixtures thereof. The vitamins also may include those exhibiting limited solubility in water, such as vitamin A compounds, and all natural and/or synthetic analogs of Vitamin A, including retinoids, carotenoids, and other compounds which possess the biological activity of Vitamin A; vitamin D compounds; vitamin E compounds, or tocopherol, including tocopherol sorbate, tocopherol acetate, other esters of tocopherol; vitamin K compounds; and mixtures thereof. In one embodiment, the compositions of the instant invention may comprise from about 0.0001% to about 10%, alternatively from about 0.001% to about 8%, alternatively from about 0.01% to about 5%, and alternatively from about 0.1% to about 1%, of the vitamin.

2. Peptides and Peptide Derivatives

The composition of the present invention may comprise one or more peptides, for example, to aid in repair of skin, to aid in exfoliation, and to deliver other benefits to the skin. Herein, “peptide” refers to peptides containing ten or fewer amino acids, their derivatives, isomers, and complexes with other species such as metal ions (for example, copper, zinc, manganese, and magnesium). As used herein, peptide refers to both naturally occurring and synthesized peptides. In one embodiment, the peptides are di-, tri-, tetra-, penta-, and hexa-peptides, their salts, isomers, derivatives, and mixtures thereof. Examples of useful peptide derivatives include, but are not limited to, peptides derived from soy proteins, palmitoyl-lysine-threonine (pal-KT) and palmitoyl-lysine-threonine-threonine-lysine-serine (pal-KTTKS, available in a composition known as MATRIXYL®), palmitoyl-glycine-glutamine-proline-arginine (pal-GQPR, available in a composition known as RIGIN®), these three being available from Sederma, France, and Cu-histidine-glycine-glycine (Cu-HGG, also known as IAMIN®).

The composition may comprise from about 1×10⁻⁷% to about 20%, alternatively from about 1×10⁻⁶% to about 10%, and alternatively from about 1×10⁻⁵% to about 5% of the peptide.

3. Sugar Amines

The composition of the present invention may comprise a sugar amine, also known as amino sugars, and their salts, isomers, tautomers and derivatives. Sugar amines can be synthetic or natural in origin and can be used as pure compounds or as mixtures of compounds (e.g., extracts from natural sources or mixtures of synthetic materials). For example, glucosamine is generally found in many shellfish and can also be derived from fungal sources. Sugar amine compounds useful in the present invention include, for example, N-acetyl-D-glucosamine, and also those described in PCT Publication WO 02/076423 and U.S. Pat. No. 6,159,485, issued to Yu, et al. In one embodiment, the composition comprises from about 0.01% to about 15%, alternatively from about 0.1% to about 10%, and alternatively from about 0.5% to about 5%, of the sugar amine.

4. Sunscreens

The composition of the present invention may comprise one or more sunscreen actives (or sunscreen agents) and/or ultraviolet light absorbers, and may be organic or inorganic. Examples of suitable sunscreen actives and ultraviolet light absorbers are disclosed in The Cosmetic, Toiletry, and Fragrance Association's The International Cosmetic Ingredient Dictionary and Handbook, 10^{th} Ed., Gottschalck, T. E. and McEwen, Jr., Eds. (2004), p. 2267 and pp. 2292-93. Particularly suitable sunscreen actives include benzophenone, benzophenone-1, benzophenone-2, benzophenone-3, benzophenone-4, benzophenone-5, benzophenone-6, benzophenone-7, benzophenone-8, benzophenone-9, benzophenone-10, benzophenone-1, benzophenone-12, benzotriazolyl dodecyl p-cresol, 3-benzylidene camphor, benzylidene camphor sulfonic acid, benzyl salicylate, bis-ethylhexyloxyphenol methoxyphenyl triazine, bornelone, bumetrizole, butyl methoxydibenzoyl-methane, butyl PABA (p-aminobenzoic acid), cinnamidopropyl-trimonium chloride, cinoxate, dea-methoxycinnamate, dibenzoxazoyl naphthalene, di-t-butyl hydroxy-benzylidene camphor, diethylamino hydroxy-benzoyl hexyl benzoate, diethylhexyl butamido triazone, diethylhexyl 2,6-naphthalate, diisopropyl ethyl cinnamate, diisopropyl methyl cinnamate, di-methoxycinnamido-propyl ethyldimonium chloride ether, dimethyl PABA ethyl cetearyldimonium tosylate, dimorpholino-pyridazinone, dimorpholino-pyridazinone, disodium bisethylphenyl triaminotriazine stilbenedisulfonate, disodium distyrylbiphenyl disulfonate, disodium phenyl dibenzimidazole tetrasulfonate, drometrizole, drometrizole trisiloxane, ethyl dihydroxypropyl PABA, ethyl diisopropyl-cinnamate, ethylhexyl bis-isopentylbenzoxazolylphenyl melamine, ethyl dimethoxybenz-ylidene dioxoimidazolidine propionate, ethylhexyl dimethyl PABA, ethylhexyl methoxy-cinnamate, ethylhexyl methoxydibenzoyl-methane, ethylhexyl salicylate, ethylhexyl triazone, ethyl methoxycinnamate, ethyl PABA, ethyl urocanate, etocrylene, 4-(2-beta-glucopyrano-siloxy) propoxy-2-hydroxybenzophenone, glyceryl ethylhexanoate dimethoxycinnamate, glyceryl PABA, glycol salicylate, hexanediol disalicylate, homosalate, isoamyl cinnamate, isoamyl p-methoxycinnamate, isopentyl trimethoxy-cinnamate trisiloxane, isopropylbenzyl salicylate, isopropyl dibenzoylmethane, isopropyl methoxy-cinnamate, kaempferia galanga root extract, menthyl anthranilate, menthyl salicylate, methoxycinnamido-propyl hydroxysultaine, methoxycinnamido-propyl laurdimonium tosylate, 4-methylbenzylidene camphor, methylene bis-benzotriazolyl tetramethylbutyl-phenol, octocrylene, octrizole, PABA, PEG-25 PABA, phenylbenzimidazole sulfonic acid, polyacrylamidomethyl benzylidene camphor, polyamide-2, polyquaternium-59, polysilicone-15, potassium methoxy-cinnamate, potassium phenyl-benzimidazole sulfonate, red petrolatum, sodium benzotriazoyl butylphenol sulfonate, sodium phenylbenz-imidazole sulfonate, sodium urocanate, TEA-phenylbenzimid-azole sulfonate, TEA-salicylate, terephthalylidene dicamphor sulfonic acid, tetrabutyl phenyl hydroxybenzoate, titanium dioxide, urocanic acid, zinc cerium oxide, zinc oxide, and mixtures thereof.

In one embodiment, the composition may comprise from about 1% to about 30%, and alternatively from about 2% to about 20% by weight of the composition, of the sunscreen active and/or ultraviolet light absorber. Exact amounts will vary depending upon the chosen sunscreen active and/or ultraviolet light absorber and the desired Sun Protection Factor (SPF) and spectrum of protection (e.g. UV-A and/or UV-B), and are within the knowledge and judgment of one of skill in the art.

5. Other Skin Care Actives

The composition of the present invention may comprise non-vitamin antioxidants, preservatives, oil control agents, flavonoids, phytosterols and/or plant hormones, protease inhibitors, tyrosinase inhibitors, anti-inflammatory agents and N-acyl amino acid compounds.

Suitable non-vitamin antioxidants include, but are not limited to, BHT (butylated hydroxy toluene), L-ergothioneine (available as THIOTANE™); tetrahydrocurcumin, cetyl pyridinium chloride, carnosine, diethylhexyl syrinylidene malonate (available as OXYNEX™), ubiquinone (co-enzyme Q10), tea extracts, including green tea extract, and combinations thereof.

Suitable examples of plant sterols and/or plant hormones include, but are not limited to, sitosterol, stigmasterol, campesterol, brassicasterol, kinetin, zeatin, and mixtures thereof.

Suitable protease inhibitors include, but are not limited to, hexamidine, vanillin acetate, menthyl anthranilate, and mixtures thereof.

Suitable anti-inflammatory agents include, but are not limited to, glycyrrhizic acid (also known as glycyrrhizin, glycyrrhixinic acid, and glycyrrhetinic acid glycoside), glycyrrhetenic acid, and combinations thereof.

Suitable N-acyl amino acid compounds include, but are not limited to, N-acyl phenylalanine, N-acyl tyrosine, their isomers, including their D and L isomers, salts, derivatives, and mixtures thereof. An example of a suitable N-acyl amino acid is N-undecylenoyl-L-phenylalanine is commercially available under the tradename SEPIWHITE® from Seppic (France).

Other useful skin care actives include dehydroepiandrosterone (DHEA), its analogs and derivatives; alpha- and beta-hydroxyacids, including glycolic acid and octanoyl salicylate, caffeine, plant extracts such as cucumber and witch hazel extract, arbutin, dimethyl aminoethanol (DMAE), kojic acid, dihydroxy acetone (DHA), soy proteins and peptides (for example, protease inhibitors such as soybean trypsin inhibitor, and Bowman-Birk inhibitor), their isomers, salts, and derivatives, and mixtures thereof

D. Conditioning Agents

The composition of the present invention may comprise from about 0.1% to about 50%, alternatively from about 0.5% to about 30%, alternatively from about 1% to about 20%, alternatively from about 2% to 15%, of a conditioning agent. These conditioning agents include, but are not limited to, hydrocarbon oils and waxes, silicones, fatty alcohol and fatty acid derivatives, cholesterol, cholesterol derivatives, diglycerides, triglycerides, vegetable oils, vegetable oil derivatives, acetoglyceride esters, alkyl esters, alkenyl esters, lanolin, wax esters, beeswax derivatives, sterols and phospholipids, salts, isomers and derivatives thereof, and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, poly alpha olefins, hydrogenated polyisobutenes and combinations thereof.

Non-limiting examples of silicone oils suitable for use herein include dimethicone copolyol, silicone cross-polymers, dimethylpolysiloxane, diethylpolysiloxane, mixed C₁₋₃₀ alkyl polysiloxanes, phenyl dimethicone, dimethiconol, and combinations thereof. In one embodiment, the silicone oils are non-volatile silicone oils selected from the group consisting of dimethicone, dimethiconol, mixed C₁₋₃₀ alkyl polysiloxanes, silicone crosspolymers, and combinations thereof. These and other examples of silicone oils useful herein are described in U.S. Pat. No. 5,011,681, issued to Ciotti et al.

Non-limiting examples of silicone cross-polymers suitable for use herein include acrylate/bis-hydroxypropyl dimethicone crosspolymer, C₃₀₋₄₅ alkyl cetearyl dimethicone crosspolymer, acrylate/bis-hydroxypropyl dimethicone crosspolymer, C₃₀₋₄₅ alkyl cetearyl dimethicone crosspolymer, cetearyl dimethicone/vinyl dimethicone crosspolymer, dimethicone crosspolymer, dimethicone crosspolymer-3, dimethicone/phenyl vinyl dimethicone crosspolymer, dimethicone/vinyl dimethicone crosspolymer, diphenyl dimethicone crosspolymer, divinyidimethicone/dimethicone crosspolymer, polyethylene glycol (PEG)-10 dimethicone crosspolymer, PEG-12 dimethicone crosspolymer, PEG-10 dimethicone/vinyl dimethicone crosspolymer, PEG-10/lauryl dimethicone crosspolymer, PEG-15/lauryl dimethicone crosspolymer, trifluoropropyl dimethicone/trifluoropropyl divinyldimethicone crosspolymer, vinyl dimethicone/lauryl dimethicone crosspolymer, vinyldimethyl/trimethylsiloxysilicate stearyl dimethicone crosspolymer, polysilicone-11, and mixtures thereof.

Other conditioning agents also useful herein are various C₁₋₃₀ monoesters and polyesters of sugars and related materials, for example, sucrose esters of fatty acids (SEFA), triglyceride esters acetoglyceride esters, alkyl esters of fatty acids having 10 to 20 carbon atoms, alkenyl esters of fatty acids having 10 to 20 carbon atoms, fatty acids having 10 to 20 carbon atoms, fatty alcohols having 10 to 20 carbon atoms, lanolin, polyhydric alcohol esters, wax esters, vegetable waxes, phospholipids, sterols, amides, isomers, salts, derivatives and mixtures thereof. These and other suitable conditioning agents are exemplified in U.S. Pat. No. 5,997,890, issued to Sine et al.

III. Methods of Use

The present invention describes methods of exfoliating mammalian skin, including delicate skin and skin surrounding the eyes, and also of simultaneously providing exfoliation and at least one additional benefit to the skin. The methods comprise the step of using a foam applicator to apply a personal care composition, both described herein, to mammalian skin. The composition may be pre-applied to the applicator, and for example delivered to the user pre-packaged as such, or the user may be instructed to apply the composition to the applicator prior to use. Alternatively, the composition may be stored in a container which is stably affixed to the applicator. The composition may be transferred to the applicator directly from the container, for example, by squeezing the container, or by other suitable means.

The composition may be applied to the skin by contacting the applicator and composition to the skin. Contact may include for example dabbing, rubbing in a circular motion, wiping, etc. on the skin with light pressure, meaning pressure that is sufficient to ensure contact between the applicator and the skin, and to ensure transfer of the composition to the skin, but which does not result in discomfort and/or undue stretching of the skin. The composition may be applied to any portion of mammalian keratinous tissue, including skin, hair, nails, lips, etc. In one embodiment, the composition is applied in need of exfoliation. In one embodiment, the composition is applied to skin surrounding the eyes, and alternatively to delicate skin.

In one embodiment of the present invention, the composition is a leave-on composition, and the method may be understood not to include the step of rinsing, wiping or otherwise removing the composition from the skin. Alternatively, the method may include the step of leaving the composition on the skin indefinitely or for a suitable period of time, for example from about 30 minutes to about 24 hours.

The amount of the composition applied, the frequency of application and the period of use will vary widely depending upon the level of components of a given composition and the level of regulation desired. For example, from about 0.01 g composition/cm² to about 1 g composition/cm² of keratinous tissue may be applied. In one embodiment, the compositions are applied at least once daily, where “daily” and “days” mean a 24-hour period. For example, the compositions may be applied daily for 30 consecutive days, alternatively for 14 consecutive days, alternatively for 7 consecutive days and alternatively for 2 consecutive days.

The method may comprise the step of inducing a temperature change in the composition and/or in the keratinous tissue either simultaneously or sequentially with the step of applying the composition. The method further may comprise additional steps which form part of a treatment or application regimen, including the steps of applying at least one additional composition, ingesting one or more dietary supplements, cleansing, etc.

IV. Article of Commerce

The present invention provides an article of commerce, comprising a foam applicator and a composition as described herein, and a communication describing the use the applicator and the composition to exfoliate delicate skin. In one embodiment, the communication describes the use of the applicator and the composition to exfoliate skin surrounding the human eyes, for example, by dabbing, rubbing and/or wiping the composition onto the skin with the applicator. The communication may be printed material attached directly or indirectly to packaging that contains the article of manufacture. Alternatively, the communication may be placed directly or indirectly near a container. Alternatively, the communication may be an electronic or a broadcast message that is associated with the applicator and/or the composition. Alternatively, the communication may describe at least one possible use, capability, distinguishing feature and/or limitation of the applicator and/or the composition. One example of a suitable communication may be printed instructions to a consumer to load the composition onto the applicator, and to gently wipe the applicator over the skin surrounding the eye. Another example of a suitable communication may be instructions to a consumer to gently wipe the applicator containing the composition over sensitive skin. The communication further may indicate to the consumer that a sufficient amount of composition is loaded onto the applicator when the composition, for example a colored composition, is visible on the applicator.

V. Kit

The present invention further provides a kit comprising at least one composition described herein. The kit may comprise an outer packaging unit, which in turn may comprise one or more inner packaging units. One non-limiting example of a suitable outer container is a box or a tray, suitable for holding a sufficient number of inner packaging units for an indicated application regimen, for example, one application per day for one month. Alternatively, the tray may contain an array of individual inner packaging units which are organized to correspond to an indicated application regimen. The kit further may comprise an implement, which may be suitable for targeted delivery of the composition to a desired area of keratinous tissue. The composition may be packaged separately from the implement, or may be contained within the implement. The kit further may comprise a plurality of components, including one or more additional compositions, one or more orally ingestible dietary supplements, an additional implement, an additional delivery enhancement device, a temperature change element, a substrate, instructions for complying with suitable application regimens, and combinations thereof.

### EXAMPLES

Examples 1-6 represent water-in-silicone emulsions suitable for application to mammalian skin by means of the applicator of the present invention, to produce an exfoliation and additional skin care benefit.

| | | Example # | | | | | |
|---|---|---|---|---|---|---|---|
| | | 1 | 2 | 3 | 4 | 5 | 6 |
| Ingredients | | (wt %) | (wt %) | (wt %) | (wt %) | (wt %) | (wt %) |
| Phase A: | | | | | | | |
| A1 | Water | qs | qs | qs | qs | qs | qs |
| A2 | Disodium EDTA | 0.05 | 0.05 | 0.05 | 0.05 | 0.05 | — |
| A3 | Niacinamide | 5.00 | 0.001 | 0.1 | 1.0 | 3.0 | 2.00 |
| A4 | Panthenol | 0.001 | 5.00 | 0.1 | 0.01 | 1.00 | — |
| A5 | Glycerin | 10.00 | 10.00 | 10.00 | 10.00 | 10.00 | 4.00 |
| A6 | Promatrixyl | 0.001 | 0.35 | 1.0 | 0.0001 | 0.1 | 0.01 |
| | (0.085% Active)⁽¹⁾ | | | | | | |
| A7 | Green Tea⁽²⁾ | 1.00 | 0.001 | 0.5 | 1.00 | 2.00 | — |
| A8 | Cucumber Extract⁽³⁾ | 1.00 | 1.00 | 1.00 | — | 1.00 | 1.00 |
| A10 | Witch hazel Extract⁽⁴⁾ | — | — | — | — | 1.00 | 1.00 |
| A11 | Veragel Liquid 1:1 | — | — | — | — | 0.15 | — |
| A12 | Caffeine | 0.50 | 0.50 | 0.50 | — | 1.00 | 0.15 |
| A13 | Propylene Glycol | 1.00 | 1.00 | 1.00 | 1.00 | 1.00 | — |
| A14 | Butylene Glycol | 1.00 | 1.00 | 1.00 | 1.00 | 1.00 | — |
| A15 | Ethylparaben | 0.10 | 0.10 | 0.10 | 0.10 | 0.10 | — |
| A16 | Propylparaben | 0.10 | 0.10 | 0.10 | 0.10 | 0.10 | — |
| A17 | Benzyl Alcohol | 0.40 | 0.40 | 0.40 | 0.40 | 0.40 | 0.40 |
| A18 | Abil EM-97⁽⁵⁾ | 0.45 | 0.45 | 0.45 | 0.45 | 0.45 | 0.30 |
| A19 | Shinetsu KF-6017⁽⁶⁾ | 0.38 | 0.38 | 0.38 | 0.38 | 0.38 | 0.25 |
| Phase B: Silicone Phase | | | | | | | |
| B1 | Dow Corning 9045⁽⁷⁾ | 14.70 | 14.70 | 14.70 | 14.70 | 14.70 | 54.10 |
| B2 | KSG-21⁽⁸⁾ | 2.50 | 2.50 | 2.50 | 2.50 | 2.50 | 0.50 |
| B3 | SF 1202⁽⁹⁾ | 18.02 | 18.02 | 18.02 | 18.02 | 18.02 | 23.45 |
| B4 | Vitamin E Acetate | 0.50 | 0.50 | 0.50 | 0.50 | 0.50 | 0.50 |
| B5 | Naturechem CR⁽¹⁰⁾ | 0.25 | 0.25 | 0.25 | 0.25 | 0.25 | — |
| B6 | Dow Corning 2503⁽¹¹⁾ | 0.50 | 0.50 | 0.50 | 0.50 | 0.50 | — |
| B7 | SEFA cottonated | 0.50 | 0.50 | 0.50 | 0.50 | 0.50 | — |
| B8 | Microthine FN-510⁽¹²⁾ | 10.00 | 10.00 | — | — | 10.00 | 10.00 |
| B9 | Micropoly 220L⁽¹³⁾ | — | — | 10.00 | — | — | — |
| B10 | EA-209⁽¹⁴⁾ | — | — | — | 2.50 | — | — |
| B11 | CM3FA70STC⁽¹⁵⁾ | 0.36 | — | — | 0.70 | 0.36 | — |
| | Product Viscosity (cps) | 40,000 | 50,600 | 58,000 | 75,000 | 47,600 | 20,800 |

Phase A Preparation:

Into a suitable container, add ingredients A1 to A12 and mix until clear. Into a separate suitable container, add ingredients A13-A17 and mix until clear. Add the A13-A17 mixture to A1-A12 mixture while mixing and heat the resulting mixture to 40-45° C. Add the ingredients A18-A19 one at a time while mixing.

Phase B Preparation:

Into a suitable container, add ingredients B1, B2 and B3 and mix until clear and homogeneous. Continue mixing, and add ingredients B4 through B7, and continue to mix until mixture is smooth. Add ingredients B8 through B11 to the mixture one at a time while continuing to mix until homogenous and smooth. Heat this mixture to 45° C.

Combining Phases:

Add the Phase B mixture to the Phase A and continue mix until the phases are inverted (i.e. the water phase is the internal phase and the silicone phase the external phase). Continue mixing for about 15 to about 30 minutes and then mill the batch for about 5 minutes at 9000 rpm using TK-25 mill. Allow the resulting composition to cool to room temperature, and transfer the batch to a suitable bulk storage container. The composition may then be transferred to containers suitable for a single application, or dose, for example, disposable containers stably affixed to an applicator as described herein.

Examples 7-11 represent silicone-in-water emulsions suitable for application to mammalian skin by means of the applicator of the present invention.

| | | Example # | | | | |
|---|---|---|---|---|---|---|
| | | 7 | 8 | 9 | 10 | 11 |
| Ingredients | | % Wt | % Wt | % Wt | % Wt | % Wt |
| Phase A: Water Phase | | | | | | |
| A1 | Water | 23.75 | 24.00 | 24.00 | 57.55 | 33.00 |
| A2 | Glycerin | 10.00 | 10.00 | 10.00 | 7.00 | 4.00 |
| A3 | Disodium EDTA | 0.05 | 0.05 | 0.05 | 0.05 | 0.05 |
| A4 | Panthenol | 0.001 | 0.1 | 1.00 | 0.05 | 2.00 |
| A5 | Green tea Extract⁽²⁾ | 1.00 | 0.500 | 0.01 | 2.0 | 0.001 |
| A6 | Cucumber Extract⁽³⁾ | 0.10 | 0.5 | 5.00 | — | 1.00 |
| A7 | Witch Hazel Extract⁽⁴⁾ | 0.10 | 0.5 | 0.001 | — | 1.00 |
| A8 | Veragel Liquid 1:1⁽²³⁾ | 0.15 | 0.15 | 0.15 | — | — |
| A9 | Promatrixyl⁽¹⁾ | 0.35 | 0.35 | 0.35 | 0.35 | 0.35 |
| A10 | Niacinamide | 5.00 | 5.00 | 5.00 | 4.00 | 2.00 |
| A11 | Caffeine | 1.00 | 1.00 | 1.00 | — | — |
| A12 | FD&C Yellow #5 | 0.0012 | 0.0012 | 0.0012 | — | — |
| A13 | FD&C Red #40 | 0.0005 | 0.0005 | 0.0005 | — | — |
| A14 | Propylene Glycol | 1.00 | 1.00 | 1.00 | 1.00 | 1.00 |
| A15 | Butylene Glycol | 1.00 | 1.00 | 1.00 | 1.00 | 1.00 |
| A16 | Ethylparaben | 0.10 | 0.10 | 0.10 | 0.10 | 0.10 |
| A17 | Propylparaben | 0.10 | 0.10 | 0.10 | 0.10 | 0.10 |
| A18 | Benzyl Alcohol | 0.40 | 0.40 | 0.40 | 0.40 | 0.40 |
| A19 | Sepigel 305⁽¹⁶⁾ | 0.50 | 0.50 | 0.50 | 0.50 | 0.75 |
| A20 | GLW75 CAP-MP⁽¹⁷⁾ | 0.35 | 0.35 | 0.35 | 0.55 | — |
| A21 | Glycosperse L-20K⁽¹⁸⁾ | 0.80 | 0.80 | 0.80 | 0.50 | 0.80 |
| Phase B: Silicone Phase | | | | | | |
| B1 | DC-9045⁽¹⁹⁾ | 14.00 | 14.00 | 12.00 | 8.00 | 22.00 |
| B2 | SF-1202⁽⁹⁾ | 17.05 | 17.05 | 15.00 | 10.00 | 15.00 |
| B3 | DC Q2-1503⁽²⁰⁾ | 3.50 | 3.50 | 3.00 | 2.00 | 3.00 |
| B4 | Dow Corning 50 cst⁽²¹⁾ | 5.20 | 5.20 | 4.50 | 3.00 | — |
| B5 | Vitamin E acetate | 0.50 | 0.50 | 0.50 | 0.50 | 0.50 |
| B6 | Brij 30⁽²²⁾ | 0.20 | 0.20 | 0.20 | 0.20 | 0.20 |
| B7 | Microthine FN510⁽¹²⁾ | 10.00 | 10.00 | 15.00 | — | 10.00 |
| Phase C: Others | | | | | | |
| C1 | Sepigel 305⁽¹⁶⁾ | 1.00 | 0.75 | 1.00 | 1.20 | 1.75 |
| | Product Viscosity (cps): | 70,000 | 43,000 | 60,000 | 20,000 | 97,000 |

Procedure:
Phase A preparation:
Into suitable container, add ingredients A1-A13. Using a propeller mix until clear. Into a second suitable container, add ingredients A14-A18. Using a magnetic stirrer, mix until clear. Add the A14-A18 ingredient mixture to the A1-A13 ingredient mixture and mix until clear. To this mixture, add ingredients A19-A21 one at a time and continue mix with a propeller mixer until Phase A is homogeneous.
Phase B preparation:
Into suitable container, add ingredients B1-B5. Using a propeller mix until homogenous and clear. Add ingredients B6 and B7 to this mixture one at a time. Continue to mix the resulting Phase B mixture until homogeneous and smooth.
Combining Phases:
Add Phase B to Phase A and mix for 10 minutes using a propeller mixer. Continue mixing, and add phase C ingredients one at a time to the Phase A/Phase B mixture. Continue mixing for 15 minutes. Mill the batch for 5 minutes using TK-50 at 8000 rpm. Transfer the batch to a suitable container.
⁽¹⁾Promatrixyl ™ Palmitoyl Pentapeptide-3 mixture (Croda Inc.)
⁽²⁾Green Tea Camellia Sinensis Leaf Extract (Fanning Inc.)
⁽³⁾Cucumber Extract Cucumis Sativus & Propylene Glycol and Water (Optima Chemical Ltd)
⁽⁴⁾Witch hazel Extract Witch Hazel Distillate (American Distilling & Manufacturing Inc.)
⁽⁵⁾Abil ™ EM-97 BIS-PEG/PPG-14/14 Dimethicone (Degussa Inc.)
⁽⁶⁾Shinetsu ™ KF-6017 Peg-10 Dimethicone (Shinetsu Inc.)
⁽⁷⁾Dow Corning ™ 9045 Dimethicone & Dimethicone Crosspolymer (Dow Corning)
⁽⁸⁾KSG-21 PEG-10 Dimethicone Crosspolymer (Shinetsu Inc.)
⁽⁹⁾SF 1202 Cyclopentasiloxane (GE or Dow Corning)
⁽¹⁰⁾Naturechem ™ CR Cetyl Ricinoleate (Caschem Inc.)
⁽¹¹⁾Dow Corning ™ 2503 Stearyl Dimethicone (Dow Corning)
⁽¹²⁾Microthine ™ FN-510 Polyethylene (Equistar Inc.)
⁽¹³⁾Micropoly ™ 220L Polyethylene (Micropowder Inc.)
⁽¹⁴⁾EA-209 Ethylene/acrylic acid Copolymer (KOBO Inc.)
⁽¹⁵⁾CM3FA70STC Titanium Dioxide SAT CR-50 mixture (KOBO Inc.)
⁽¹⁶⁾Sepigel ™ 305 Polyacrylamide (Seppic Inc.)
⁽¹⁷⁾GLW75 CAP-MP Titanium Dioxide (KOBO Inc.)
⁽¹⁸⁾Glycosperse ™ L-20K Polysorbate-20 (Lonza Ltd.)
⁽¹⁹⁾Dow Corning ™ 9045 Dimethicone & Dimethicone Crosspolymer (Dow Corning)
⁽²⁰⁾Dow Corning ™ Q2-1503 Polydimethylsiloxane (Dow Corning)
⁽²¹⁾Dow Corning ™ 50 cst Dimethicone fluid (Dow Corning)
⁽²²⁾Brij ™ 30 Laureth-4 (Uniquema Inc.)
⁽²³⁾Pure World Botanicals, Inc.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

Whereas particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of exfoliating mammalian skin comprising the step of using a foam applicator to apply a personal care composition to delicate mammalian skin, wherein:
a) the foam applicator comprises a material selected from the group consisting of polyester, polyurethane, polyether, polyethylene, polystyrene, cross-linked polyethylene, and mixtures thereof, and has a porosity of from about 10 to about 110 pores per linear inch;
b) the personal care composition comprises a dermatologically-acceptable carrier in the form of an emulsion.

2. The method of claim 1, wherein the skin surrounds the human eye.

3. The method of claim 1, wherein the foam applicator has a thickness of from about 2 mm to about 8 mm.

4. The method of claim 1, wherein the emulsion is a silicone-in-water emulsion.

5. The method of claim 1, wherein the emulsion is a water-in-silicone emulsion.

6. The method of claim 1, wherein the personal care composition comprises from about 0.1% to about 20% of a particulate material.

7. The method of claim 1, wherein the personal care composition is in a container stably affixed to the foam applicator.

8. The method of claim 7, wherein the container comprises from about 0.1 ml to about 5 ml of the personal care composition.

9. The method of claim 1, wherein the composition is visually distinct from the applicator.

10. A method of exfoliating mammalian skin and of simultaneously providing an additional benefit to the skin comprising the step of using a foam applicator to apply a personal care composition to delicate mammalian skin, wherein:
a) the foam applicator comprises a material selected from the group consisting of polyester, polyurethane, polyether, polyethylene, polystyrene, cross-linked polyethylene, and mixtures thereof, and has a porosity of from about 10 to about 110 pores per square inch;
b) the personal care composition comprises a dermatologically-acceptable carrier in the form of an emulsion and at least one additional skin benefit agent.

11. The method of claim 10, wherein the emulsion is a silicone-in-water emulsion.

12. The method of claim 10, wherein the skin benefit agent is selected from the group consisting of vitamin B compounds, vitamin C compounds, peptides, sugar amines, caffeine, plant extracts, anti-inflammatory agents, protease inhibitors, and mixtures thereof.

13. The method of claim 10, wherein the skin benefit agent is selected from the group consisting of niacinamide, palmitoyl-lysine-threonine-threonine-lysine-serine, ascorbyl glucoside, N-acetyl glucosamine, cucumber extract, caffeine, witch hazel extract, aloe vera, tetrahydrocurcumin, glycyrhettinic acid, hexamidine, their isomers, salts and derivatives, and mixtures thereof.

14. The method of claim 10, wherein the personal care composition comprises from about 0.1% to about 20% of a particulate material.

15. The method of claim 10, wherein the composition is visually distinct from the applicator.

16. The method of claim 10, wherein the benefit is selected from the group consisting of wrinkle reduction, reduction of discoloration, reduction of puffiness, improving elasticity, and combinations thereof.

17. The method of claim 10, wherein the skin surrounds the human eye.

18. An article of commerce comprising:
a) a foam applicator comprising a material selected from the group consisting of polyester, polyether, polyethylene, polystyrene, cross-linked polyethylene, and mixtures thereof, and has a porosity of from about 10 to about 110 pores per linear inch;
b) the personal care composition comprising a dermatologically-acceptable carrier in the form of an emulsion; and
c) a communication directing a consumer to apply the composition to delicate skin to provide an exfoliation benefit.

19. The article of commerce of claim 18, wherein the consumer is directed to apply the composition to skin surrounding the eyes.

20. A kit comprising:
a) a foam applicator comprising a material selected from the group consisting of polyester, polyether, polyethylene, polystyrene, cross-linked polyethylene, and mixtures thereof, and has a viscosity of from about 10 to about 110 pores per linear inch;
b) the personal care composition comprising a dermatologically-acceptable carrier in the form of an emulsion; and
c) at least one additional component selected from the group consisting of at least one additional composition, an orally ingestible dietary supplement, an additional implement, an additional delivery enhancement device, a temperature change element, an additional substrate, instructions for complying with suitable application regimens, and combinations thereof.
